(19) United States
(12) Patent Application Publication
(10) Pub. No.: US 2026/0232209 A1
(43) Pub. Date: Aug. 13, 2026

(54) INDWELLING SENSOR

(71) Applicant: Certus Critical Care, Inc., Salt Lake City, UT (US)

(72) Inventors: Samuel LARSON, Bellingham, WA (US); David I. POISNER, Carmichael, CA (US); Erik S. LIDDIARD, Eagle Mountain, UT (US)

(21) Appl. No.: 19/310,832

(22) Filed: Aug. 26, 2025

Related U.S. Application Data
(60) Provisional application No. 63/687,291, filed on Aug. 26, 2024.

(51) Int. Cl. A61B 5/0215; A61B 5/00; A61B 5/0205; A61B 5/021; A61B 5/145

(52) U.S. Cl. CPC A61B 5/02156; A61B 5/0205; A61B 5/02141; A61B 5/02152; A61B 5/14503; A61B 5/14542; A61B 5/14546; A61B 5/6831; A61B 5/6847; A61B 5/6876; A61B 5/742; A61B 2560/0223; A61B 2560/0242; A61B 2560/0462; A61B 2560/06

(57) ABSTRACT

Described herein are systems, methods, and kits for measuring various physiological parameters from within the body (e.g. from within the vasculature). The systems and methods may include one or more sensors that may be calibrated using valves, ports, and/or a body-worn sensor. The information measured by the indwelling sensors may be wirelessly transmitted to an external monitor.

### CROSS-REFERENCE TO RELATED APPLICATIONS

This application claims priority to U.S. Provisional Application No. 63/687,291, filed on Aug. 26, 2024, which is hereby incorporated by reference in its entirety.

### GOVERNMENT SUPPORT

This invention was made with government support under Department of Defense USA Medical Research Acquisition Activity grant W81XWH-22-C-0044. The government has certain rights in the invention.

### FIELD

This application generally relates to systems, methods, and kits for measuring various physiological parameters, including blood pressure, from within the vasculature. The systems, methods, and kits may include one or more sensors that may obtain more accurate readings during patient and/or sensor movement. The information measured by the indwelling sensors may be wirelessly transmitted to an external monitor.

### BACKGROUND

Blood pressure may be measured in a variety of ways during patient care. For example, a common way of measuring blood pressure employs a blood pressure cuff, which is wrapped around an arm or a leg to control the flow of blood through the arteries. The cuff may be inflated until the artery is fully occluded, and then blood pressure is measured as the pressure in which flow is first heard through the vessel (systolic blood pressure) and then the pressure when that flow is no longer heard (diastolic blood pressure). Other methods of measuring blood pressure may include invasive arterial blood pressure monitoring, which uses a catheter placed inside an artery that is connected to a non-expandable piece of tubing. When the tubing and catheter are filled with fluid, the pressure from the fluid transmits the pressure from inside the blood vessel to a pressure sensor residing outside of the body. Another method may include estimating blood pressure at the fingertip of a patient using a technique called photoplethysmography, in which light is passed through the skin of a finger and the reflected and/or transmitted light analyzed to determine blood pressure.

Continuously measuring blood pressure with every heartbeat is generally important for patients who are critically ill. In this instance, an arterial line may be placed for invasive arterial pressure monitoring. While this technology has been used for decades, there are significant drawbacks to the system given that there are several steps during setup that may result in inaccurate measurement readings. For example, the arterial line may become over dampened (e.g., due to low infusion bag pressure, kinks in the tubing, blood clots and/or air bubbles in the tubing), or under dampened (e.g., due to inappropriate pressurization from the pressure bag, over stiff tubing, kinks in the tubing). Another common problem is that the sensor outside the body may be set and/or zeroed at a level that is not aligned with the heart, resulting in pressures that are higher or lower than the patient's actual blood pressure. This may occur when the initial setup was inaccurate or because of patient movement after the line is set up. The actual setup of the arterial line is also complex and cumbersome. A tubing set must be fully flushed with saline prior to use because air bubbles in the tubing may result in inaccurate blood pressure readings, as mentioned above. Additionally, the arterial line must be continuously flushed throughout use, otherwise blood will clot in the catheter and result in inaccurate blood pressure readings, as also mentioned above. Therefore, a pressurized bag of saline is needed while the arterial line is in the patient. Finally, invasive arterial pressure monitoring is very sensitive to any movement of the patient or the line. Any tapping on the line or excessive movement of the line may produce artifacts in the blood pressure tracing, which may further result in an inaccurate blood pressure reading.

Accordingly, it would be beneficial to have sensor systems for measuring blood pressure and other physiological parameters that have a simpler setup, and which are less sensitive to patient or arterial line movement. It would also be useful to have sensor systems capable of accurately measuring physiological parameters without the sensor being continuously at the same level as the heart. Sensor systems capable of coupling with other devices for transmission of physiological parameter data would also be useful.

### SUMMARY

Described herein are sensor systems, methods, and kits that may measure blood pressure and other physiological parameters of a patient from one or more sensors positioned within the vasculature, e.g., an artery or a vein. In some instances, the vasculature is the peripheral vasculature such as a peripheral artery or a peripheral vein. By placing the one or more sensors within the body, the problems associated with conventional arterial lines, e.g., complex setup that may result in inaccurate measurements and tedious maintenance (e.g., due to constant line flushing), as discussed above, may be prevented, and thus provide more accurate measurements of physiological parameters. When blood pressure is to be measured, the systems may also include a component configured to recalibrate the sensor due to changes in patient movement and/or sensor positioning with respect to the level of the heart of the patient. As used herein, the term “indwelling” is used to describe one or more sensors placed within the body, e.g., a blood vessel.

In general, the systems for measuring physiological parameters of a patient may include a measurement unit comprising a housing and a sensor line unit releasably coupled to the housing.

The sensor line unit may include an elongate member having one or more sensors along a length thereof configured to measure at least one physiological parameter from within a blood vessel. The one or more sensors may be a solid-state sensor as described in U.S. Publication No. 2023/0380702. In one variation, the one or more sensors may be a blood pressure sensor. Some variations of the sensor line unit may be configured to advance the elongate member to position the one or more sensors within the blood vessel, and retract the elongate member to position the one or more sensors outside of the blood vessel. The measurement unit and the sensor line unit may be reusable or disposable.

Additionally, the systems may include a valve assembly coupled to a sheath concentrically disposed over the elongate member. Instead of being coupled to the sheath, in some instances the valve assembly may be coupled to the sensor line (e.g., a catheter or tubing extending from the measurement unit and through which the elongate member is advanced and retracted), or both the sensor line and the sheath. The valve assembly may include one or more of a first valve or a second valve. In some variations, the valve assembly may comprise a first valve and a port configured to expose the one or more sensors to the environment (e.g., by removing a cap covering the port). A seal disposed between the first valve and the port, or the first valve and a second valve may allow blood to be drawn from the first valve, e.g., when a distal tip of the elongate member is positioned between the first valve and the port or between the first valve and the second valve. The first valve may also be used to introduce fluids for flushing or to increase circulatory volume, and/or to deliver medications to the patient. In these instances, the port or the second valve may be configured to calibrate the one or more sensors when the one or more sensors is positioned at the port or the second valve. The fluids that may be delivered via the first valve include without limitation, one or more of normal saline, D5W, lactated Ringer's solution, Plasmalyte, plasma, albumin, blood, or a blood product. The medications that may be delivered via the first valve may include without limitation, one or more of norepinephrine, epinephrine, vasopressin, phenylephrine, dopamine, dobutamine, angiotensin II, clevidipine, nifedipine, labetalol, esmolol, fenoldopam, nitroglycerin, or nitroprusside.

Instead of a valve assembly, in some variations, one or more connectors including one or more ports may be employed to couple the sensor line and/or sheath to the access catheter, and the one or more ports may be used to calibrate the one or more sensors. In these variations, the one or more ports may be configured to expose the one or more sensors to the environment by removing a cap covering the port. In one variation, the system may include two ports, a first port for calibration and a second port for blood sampling, delivery of fluids, and/or delivery of medications. Any of the ports (e.g., a first port, a second port) and/or valves (e.g., a first valve, a second valve) may be used to calibrate the one or more sensors, introduce a medication or other fluid into the patient, and/or provide a fluid for flushing a portion of the system, e.g., the elongate member, the access catheter, and/or the sheath. The fluids that may be delivered via the one or more ports include without limitation, one or more of normal saline, D5W, lactated Ringer's solution, Plasmalyte, plasma, albumin, blood, or a blood product. The medications that may be delivered via the one or more ports may include without limitation, one or more of norepinephrine, epinephrine, vasopressin, phenylephrine, dopamine, dobutamine, angiotensin II, clevidipine, nifedipine, labetalol, esmolol, fenoldopam, nitroglycerin, or nitroprusside.

Additionally or alternatively, a separate body-worn sensor (e.g., including an adhesive patch) may be used to calibrate the one or more indwelling sensors as described further below. An access catheter configured to be introduced into a blood vessel may be coupled to the valve assembly. In some variations, a proximal portion of the sheath may be made from a translucent material so that a marker at a proximal end of the elongate member may be visualized, and advancement and retraction of the elongate member tracked by the user.

The systems may also include a display on the measurement unit that may or not be a touchscreen. The display may be configured to visually present data relating to the measured at least one physiological parameter, instructions for setup of the system, an error in function of the measurement unit, sensor line unit, one or more sensors, or a combination thereof. A controller configured to wirelessly communicate the measured at least one physiological parameter with one or more devices may be included in the measurement unit.

The sensor line unit may include a cartridge configured to couple with the housing of the measurement unit, where the cartridge may include an actuator configured to advance and retract the elongate member. In some variations, the actuator may include a wheel, an actuator housing, and space therebetween configured prevent over tensioning of the elongate member during retraction of the elongate member. The elongate member of the sensor line unit may include a tube having a lumen and a plurality of laser cut openings on a distal portion thereof. In one variation, the tube comprises nitinol. However, the tube may be made from other metals, metal alloys, or polymeric materials.

The diameter of the elongate member may be between about 0.6 mm and about 4.6 mm, including all values and sub-ranges therein. For example, the diameter of the elongate member may be about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm, about 3.0 mm, about 3.1 mm, about 3.2 mm, about 3.3 mm, about 3.4 mm, about 3.5 mm, about 3.6 mm, about 3.7 mm, about 3.8 mm, about 3.9 mm, about 4.0 mm, about 4.1 mm, about 4.2 mm, about 4.3 mm, about 4.4 mm, about 4.5 mm, or about 4.6 mm. In one variation, the diameter of the elongate member may be about 0.64 mm. In some variations, the elongate member may include a window configured to expose the sensor to blood within the blood vessel.

The systems described herein may be configured to measure at least one physiological parameter, such as, but not limited to, blood pressure, mean arterial blood pressure, a pH level, a lactate level, a carbon dioxide level, and an oxygen level. In one variation, the at least one physiological parameter is blood pressure. The at least one physiological parameter may be measured while the one or more sensors are positioned within a blood vessel, e.g., an artery or a vein.

Some variations of the systems for measuring physiological parameters of a patient may include at least a first sensor configured to measure at least one physiological parameter from within a blood vessel, and a wearable sensor configured to calibrate the first sensor. The first sensor may be attached to an elongate member of a sensor line unit that may be releasably coupled to a measurement unit. The first sensor may be configured to measure a physiological parameter, e.g., blood pressure, from inside a blood vessel such as an artery or a vein. In some variations, the first sensor may be a solid-state sensor. The wearable sensor may include an adhesive layer to aid in securing the wearable sensor to a portion of the patient's body (e.g., on the chest, at the level of the patient's heart). The wearable sensor may include a barometer.

Methods of measuring physiological parameters of a patient are also described herein. The method may generally include positioning an elongate member of a sensor line unit comprising one or more sensors into a blood vessel, coupling the sensor line unit to a measurement unit, and measuring at least one physiological parameter of the patient. The elongate member may be advanced to position the one or more sensors within the blood vessel and retracted to position the one or more sensors outside of the blood vessel.

The methods may also include releasably attaching the measurement unit to a limb of the patient. The limb may be an arm (e.g., a wrist) or a leg of the patient. In some instances, the method may further include coupling a valve assembly to the sensor line and/or a sheath concentrically disposed over the elongate member. The elongate member comprising the one or more sensors may be advanced through the valve assembly and an access catheter into the blood vessel.

When the elongate member is retracted, a blood sample may be drawn from the patient via a first valve of the valve assembly. Thereafter, the elongate member may be re-advanced to position the one or more sensors through the access catheter and into the blood vessel again to continue measuring the one or more physiological parameters, e.g., blood pressure. Retraction and advancement of the elongate member and associated sensors may be repeated as many times as desired. The methods may further include calibrating the one or more sensors using a port and/or a valve (e.g., a second valve) of the valve assembly. In this instance, the port and/or the valve (e.g., a second valve) may be open to atmospheric pressure. For example, the port may be covered with a cap when calibration is not being performed, and the cap may be removed when calibration is to be conducted. Other variations of the method may utilize a body-worn sensor to adjust or correct the sensor reading in response to changes in the position of the patient. For example, if the patient is raised up from lying to seated and the level of the heart increases, then the body-worn sensor may correct for that movement. In another example, if the arm of the patient moves down or up then the body-worn sensor may correct for that movement using the body-worn sensor.

The methods described herein may measure at least one physiological parameter of a patient, such as, but not limited to, blood pressure, mean arterial blood pressure, a pH level, a lactate level, a carbon dioxide level, and an oxygen level. In one variation, the at least one physiological parameter is blood pressure. The at least one physiological parameter may be measured while the one or more sensors are positioned within a blood vessel such as an artery or a vein. For example, the blood vessel may be a radial artery, a subclavian artery, a femoral artery, the aorta, a carotid artery, a femoral vein, a subclavian vein, a jugular vein, or the vena cava.

Kits for measuring at least one physiological parameter of a patient are also described herein. The kits may comprise one or more sensor line units, where the sensor line units may include an elongate member having one or more sensors along a length thereof configured to measure at least one physiological parameter from within a blood vessel. The sensor line units may also be configured to advance the elongate member to position the one or more sensors within the blood vessel, and retract the elongate member to position the one or more sensors outside of the blood vessel. The kits may further include one or more of a measurement unit, a valve assembly, an access catheter, or a body-worn sensor.

### BRIEF DESCRIPTION OF THE DRAWINGS

FIG. 1A is a side perspective view of an exemplary system for measuring physiological parameters including a measurement unit, a sensor line unit, an elongate body having a sensor coupled thereto, and a valve assembly including a plurality of valves, each valve configured to flush, draw blood, and/or calibrate the sensor to atmospheric pressure.

FIGS. 1B and 1C are side perspective views of another exemplary system for measuring physiological parameters including a valve assembly comprising a valve and a capped port. The port is capped in FIG. 1B and uncapped in FIG. 1C.

FIG. 2 is an exploded view of the components of another exemplary system for measuring physiological parameters.

FIG. 3 shows an exemplary system with the sensor line unit decoupled from the measurement unit.

FIG. 4A is an exploded view of the components of an exemplary elongate member.

FIG. 4B is an exemplary sheath that may be disposed over at least a portion of the elongate member.

FIGS. 5A and 5B illustrate the position of an exemplary visual marker when the elongate member is extended (FIG. 5A), and when the elongate member is retracted (FIG. 5B).

FIGS. 6A and 6B are side perspective views of an exemplary system showing the elongate member extended from an access catheter (FIG. 6A), and retracted into the access catheter (FIG. 6B).

FIGS. 7A and 7B show the position of an exemplary elongate member, sensor, and seal with respect to an exemplary valve assembly. FIG. 7A depicts the elongate member and sensor retracted to a position that may allow calibration of the sensor, and FIG. 7B depicts the elongate member extended such that fluid flow is prevented through the valve assembly.

FIGS. 8A-8C show the internal components of an exemplary cartridge of a sensor line unit

### DETAILED DESCRIPTION

Described herein are sensor systems, methods, and kits that may measure various physiological parameters, e.g., blood pressure, of a patient from one or more sensors positioned within the body (e.g., within the vasculature such as within an artery or a vein). As previously mentioned, by placing the one or more sensors within the body, the problems associated with conventional arterial lines, e.g., complex setup that may result in inaccurate measurements and tedious maintenance (e.g., due to constant line flushing), may be prevented, and thus provide more accurate measurements of physiological parameters.

When blood pressure or other physiological parameter is to be measured, the systems may include a component configured to recalibrate the sensor due to changes in patient movement and/or sensor positioning with respect to the level of the heart of the patient. Such systems may be used with conventional arterial lines and/or sensors positioned within the vasculature as described herein. The systems, methods, and kits described here may monitor the blood pressure of a patient continuously or intermittently. In addition to blood vessels, the systems, methods, and kits may also be used to measure the pressure in the body of a muscle, in the abdominal cavity, or any location in which continuous and/or intermittent pressure measurements may be desired.

### Systems

The sensing systems described herein may overcome the limitations of setup and continuous flushing that must be maintained when using fluid column sensing by, for example, using one or more indwelling sensors (e.g., intravascularly placed sensors). Traditionally, a pressure column sensing system for measuring blood pressure requires a pressurized bag of saline or other fluid which slowly flushes the line and catheter with 3-5 ml/hr so that blood does not clot in the catheter. This creates an opportunity for air bubbles to be entrained in the fluid lines, which may dampen the signal transduction from the blood vessel back to the sensor. By placing the sensor in the vessel itself and sealing the catheter in the process, the chance of blood clots developing within the catheter may be minimized. Furthermore, fluid column pressure sensing may not compensate for changes in the elevation of the measurement point, such as when a patient's wrist is moved or the patient is rolled on their side. The use of an auxiliary sensor such as a wearable sensor (e.g., a body-worn sensor such as an adhesive patch), as further described below, may increase the accuracy of the measured pressures from the one or more indwelling sensors.

The systems for measuring physiological parameters of a patient may include a measurement unit comprising a housing and a sensor line unit releasably coupled to the housing.

The sensor line unit may include an elongate member having one or more sensors along a length thereof configured to measure at least one physiological parameter from within a blood vessel. The one or more sensors may be a solid-state sensor as described in U.S. Publication No. 2023/0380702, which is hereby incorporated by reference herein in its entirety. Additionally, the systems may include a valve assembly coupled to a sensor line of the sensor line unit and/or a sheath disposed over (e.g., concentrically) the elongate member. The valve assembly may be used to calibrate the one or more sensors, obtain a blood sample from the patient, deliver a fluid (e.g., for flushing a portion of the system or to increase circulatory volume of the patient), and/or to deliver a medication to the patient. An access catheter configured to be introduced into a blood vessel may be coupled to the valve assembly. The systems may further include a wearable sensor (e.g., a body-worn sensor) to assist with indwelling (e.g., intravascular) sensor calibration.

### Measurement Unit

The measurement unit of the systems described herein may generally include one or more processors and other electronics configured to convert the signal measured by the one or more sensors in the vasculature to a physiological parameter value, e.g., a value indicative of blood pressure, mean arterial blood pressure, a pH level, a lactate level, a carbon dioxide level, and/or an oxygen level. In one variation, the at least one physiological parameter value is indicative of blood pressure. The measurement unit may include a user interface (e.g., screen) configured to display or otherwise communicate the physiological parameter to a user. In some variations, the measurement unit may be configured to transmit the measured sensor signals to a bedside monitor either wirelessly or via a wired connection. Additionally or alternatively, the measurement unit may be configured (e.g., sized) for attachment to a part of the patient, e.g., an arm or a leg. For example, the width of the measurement unit may range from about 2.5 cm to about 12.7 cm, including all values and sub-ranges therein, and the length of the measurement unit may range from about 2.5 cm to about 12.7 cm, including all values and sub-ranges therein. For example, the width of the measurement unit may be about 2.5 cm, about 3.0 cm, about 3.5 cm, about 4.0 cm, about 4.5 cm, about 5.0 cm, about 5.5 cm, about 6.0 cm, about 6.5 cm, about 7.0 cm, about 7.5 cm, about 8.0 cm, about 8.5 cm, about 9.0 cm, about 9.5 cm, about 10.0 cm, about 10.5 cm, about 11.0 cm, about 11.5 cm, about 12.0 cm, or about 12.5 cm. In one variation, the width of the measurement unit is about 6.2 cm (about 2.4 inches). With respect to the length of the measurement unit, it may be about 2.5 cm, about 3.0 cm, about 3.5 cm, about 4.0 cm, about 4.5 cm, about 5.0 cm, about 5.5 cm, about 6.0 cm, about 6.5 cm, about 7.0 cm, about 7.5 cm, about 8.0 cm, about 8.5 cm, about 9.0 cm, about 9.5 cm, about 10.0 cm, about 10.5 cm, about 11.0 cm, about 11.5 cm, about 12.0 cm, or about 12.5 cm. In one variation, the length of the measurement unit may be about 8.8 cm (about 3.5 inches). The measurement unit may have a depth ranging from about 2.5 cm to about 5.5 cm, including all values and sub-ranges therein. For example, the depth of the measurement unit may be about 2.5 cm, about 3.0 cm, about 3.5 cm, about 4.0 cm, about 4.5 cm, about 5.0 cm, or about 5.5 cm. In one variation, the depth of the measurement unit may be about 2.8 cm (about 1.1 inches). The measurement unit may include a user interface configured for user input and/or to calibrate the one or more sensors, as further described below, and may be configured to work via battery power or via a wall outlet.

The measurement unit may include a housing, which may comprise a housing body and a cover. The housing body and the cover may be attached by any suitable means, such as for example, mechanical attachment mechanisms (e.g., a friction fit, interlocking elements, tabs), adhesive, a combination thereof, or other attachment mechanisms. The housing body may contain one or more circuit boards (e.g., mounted inside the housing body). When batteries are used, they may be disposed within the housing body and furnished as a replaceable battery pack. The measurement unit may be reusable or disposable (i.e., single-use).

### Sensor Line Unit

The sensor line unit of the systems described herein may be coupled to the measurement unit and may generally comprise a cartridge and an elongate member extending from the cartridge. The elongate member may include one or more sensors disposed along a length thereof. The sensors may measure one or more physiological parameters such as, but not limited to, blood pressure, mean arterial blood pressure, pH level, lactate level, carbon dioxide level, or oxygen level. In some variations, the one or more sensors may be a solid-state sensor, as mentioned above. The sensor line unit may be generally configured to advance the elongate member to position the one or more sensors within the body (e.g., a blood vessel), and/or retract the elongate member to position the one or more sensors outside of the body. One or more markers may be included on the elongate member to visually track the position of the elongate member during advancement and retraction. The sensor line unit may be reusable or disposable (i.e., single-use).

The elongate member may comprise an elongate tube or wire including one or more sensors. The one or more sensors may be embedded within a portion of the elongate tube or wire, or coupled to the elongate tube or wire. The one or more sensors may be positioned on any portion of the elongate tube or wire (e.g., a distal portion of the elongate tube or distal portion of the wire), and may be sized for advancement and/or retraction through an access catheter and into the body. The length of the elongate member may range from about 40 cm to about 75 cm, including all values and sub-ranges therein. For example, the length of the elongate member may be about 40 cm, about 45 cm, about 50 cm, about 55 cm, about 60 cm, about 65 cm, about 70 cm, or about 75 cm. The diameter of the elongate member may be about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm, about 3.0 mm, about 3.1 mm, about 3.2 mm, about 3.3 mm, about 3.4 mm, about 3.5 mm, about 3.6 mm, about 3.7 mm, about 3.8 mm, about 3.9 mm, about 4.0 mm, about 4.1 mm, about 4.2 mm, about 4.3 mm, about 4.4 mm, about 4.5 mm, or about 4.6 mm. In one variation, the diameter of the elongate member may be about 0.64 mm. In some variations, the elongate member may include a window configured to expose the sensor to blood within the blood vessel.

The tube or wire of the elongate member may be comprised of nitinol or other metals, metal alloys, or polymeric materials. In some variations, the elongate member may include a plurality of laser cut openings on a distal portion thereof to enhance flexibility of the distal portion. Additionally or alternatively, the elongate member may be configured to include an atraumatic tip (e.g., a blunt and/or rounded tip). The elongate member may reside and be slidable within the sensor line of the sensor line unit. A proximal end of the sensor line may be coupled to the cartridge via a friction fit or threaded connector, or other suitable connector. In some variations, a sheath may be disposed about (e.g., concentrically, eccentrically) the elongate member.

The cartridge of the sensor line unit may include a housing and a cartridge wheel about which the elongate member is wound. Rotation of the cartridge wheel in one direction may advance the elongate member from the housing through the access catheter and into the body, e.g., the vasculature, and rotation of the cartridge wheel in the opposite direction may retract the elongate member back towards the housing. A space between the housing and the cartridge wheel may allow the elongate member to be wound around an inside edge of the cartridge wheel during retraction of the elongate member and along an outside edge of the cartridge wheel during advancement of the elongate member. The size of the space may range from about 4.0 square millimeters to about 5.0 square millimeters, including all values and sub-ranges therein. For example, the size of the space may be about 4.0 square millimeters, about 4.1 square millimeters, about 4.2 square millimeters, about 4.3 square millimeters, about 4.4 square millimeters, about 4.5 square millimeters, about 4.6 square millimeters, about 4.7 square millimeters, about 4.8 square millimeters, about 4.9 square millimeters, or about 5.0 square millimeters. In one variation, the size of the space is about 4.4 square millimeters. The two edges may be configured to both rotate with the wheel to prevent the elongate member from binding (e.g., twisting, snagging on portions of the elongate member looped around the wheel, having uneven tension) during winding (retraction) and unwinding (advancement).

### Valve Assembly

The systems described herein may include a valve assembly disposed between the sensor line of the sensor line unit and the access catheter. One or more connectors may be employed, which may be configured to couple the valve assembly to the sensor line and the access catheter. In some variations, the valve assembly may also be coupled to a sheath disposed over (e.g., concentrically, eccentrically) the elongate member.

In some variations, the valve assembly may comprise a plurality of valves (e.g., two, three, four, or more). For example, the valve assembly may include at least a first valve and a second valve. The valve assembly may further comprise a seal (e.g., an O-ring) disposed between the first valve and the second valve, which may allow blood to be drawn from the first valve, e.g., when a distal tip of the elongate member is positioned between the first and second valves. In this instance, the second valve may be configured to calibrate the one or more sensors to atmospheric pressure when the one or more sensors is positioned at the second valve. Additionally or alternatively, a separate body-worn sensor (e.g., including an adhesive patch) may be used to calibrate the one or more indwelling vascular sensors as described further below or to adjust/correct the measurement (e.g., pressure measurement) if the patient position is altered. The first valve may also be configured for coupling to a source of fluid for flushing and/or to a source of medication or other fluid for delivery to the patient. The valve via which a blood sample may be drawn or fluids and/or medications or other fluids delivered may be a valve on a distal portion of the valve assembly (e.g., closer to the access catheter), and the valve used for calibration may be a valve on a proximal portion of the valve assembly (e.g., closer to the measurement unit), although in some variations the functions of the distal and proximal valves may be interchanged, e.g., when calibration and blood sampling, fluid and/or medication delivery is not performed at the same time. For example, the valve on the distal portion of the valve assembly is used for calibration and the valve on the proximal portion of the valve assembly may be used to draw a blood sample and/or deliver fluids and/or medications or other fluids. In some variations, the valve assembly may comprise additional (e.g., third, fourth, etc.) valves configured for coupling to a source of fluid for flushing and/or to a source of medication or other fluid for delivery to the patient.

The fluids that may be delivered via one or more of the valves include without limitation, one or more of normal saline, D5W, lactated Ringer's solution, Plasmalyte, plasma, albumin, blood, or a blood product. The medications that may be delivered via the one or more valves may include without limitation, one or more of norepinephrine, epinephrine, vasopressin, phenylephrine, dopamine, dobutamine, angiotensin II, clevidipine, nifedipine, labetalol, esmolol, fenoldopam, nitroglycerin, or nitroprusside.

In other variations, the valve assembly may comprise a port. For example, the valve assembly may comprise a valve (e.g., a first valve), a connector with a port, and a cap configured to cover the port. Similar to the instance of the valve assembly including first and second valves, the valve assembly may comprise a seal (e.g., an O-ring) disposed between the first valve and the port, which may allow blood to be drawn from the first valve, e.g., when a distal tip of the elongate member is positioned between the first valve and the port. In this instance, the port may be configured to calibrate the one or more sensors to atmospheric pressure when the one or more sensors is positioned at the port. Additionally or alternatively, a separate body-worn sensor (e.g., including an adhesive patch) may be used to calibrate the one or more indwelling sensors as described further below or to adjust/correct the measurement (e.g., pressure measurement) if the patient position is altered. The port may also be configured for coupling to a source of fluid for flushing and/or to a source of medication or other fluid for delivery to the patient. In some variations, the valve assembly may comprise a plurality of valves (e.g., two, three, four or more) and a port.

### Access Catheter

The systems described herein may also comprise an access catheter. The access catheter may be configured such that at least a portion (e.g., at least a distal portion) may be introduced into a body (e.g., vasculature) of a patient. The access catheter may be coupled to the valve assembly via a hub at a proximal end of the access catheter. The access catheter may generally comprise any catheter configured for placement within the body (e.g., catheters configured for intravenous or intra-arterial use), and may have a lumen through which the elongate member may be advanced and retracted.

The length of the access catheter may range from about 3.0 cm to about 25.0 cm, including all values and sub-ranges therein. For example, the length of the access catheter may be about 3.0 cm, about 3.5 cm, about 4.0 cm, about 4.5 cm, about 5.0 cm, about 5.5 cm, about 6.0 cm, about 6.5 cm, about 7.0 cm, about 7.5 cm, about 8.0 cm, about 8.5 cm, about 9.0 cm, about 9.5 cm, about 10.0 cm, about 10.5 cm, about 11.0 cm, about 11.5 cm, about 12.0 cm, about 12.5 cm, about 13.0 cm, about 13.5 cm, about 14.0 cm, about 14.5 cm, about 15.0 cm, about 15.5 cm, about 16.0 cm, about 16.5 cm, about 17.0 cm, about 17.5 cm, about 18.0 cm, about 18.5 cm, about 19.0 cm, about 19.5 cm, about 20 cm, about 20.5 cm, about 21 cm, about 21.5 cm, about 22 cm, about 22.5 cm, about 23 cm, about 23.5 cm, about 24 cm, about 24.5 cm, or about 25 cm. The diameter of the access catheter may range from about 24.0 gauge to about 20.0 gauge, including all values and sub-ranges therein. For example, the diameter of the access catheter may be about 24 gauge, about 23.5 gauge, about 23.0 gauge, about 22.5 gauge, about 22.0 gauge, about 21.5 gauge, or about 20.0 gauge.

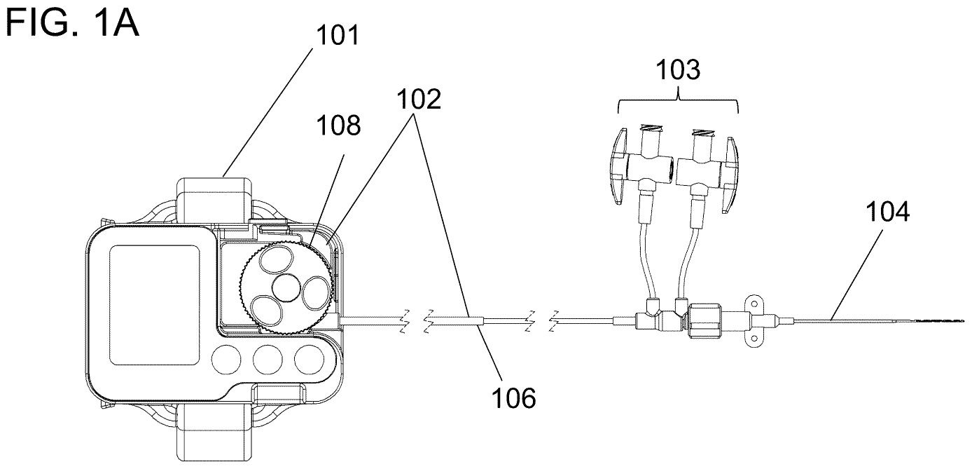

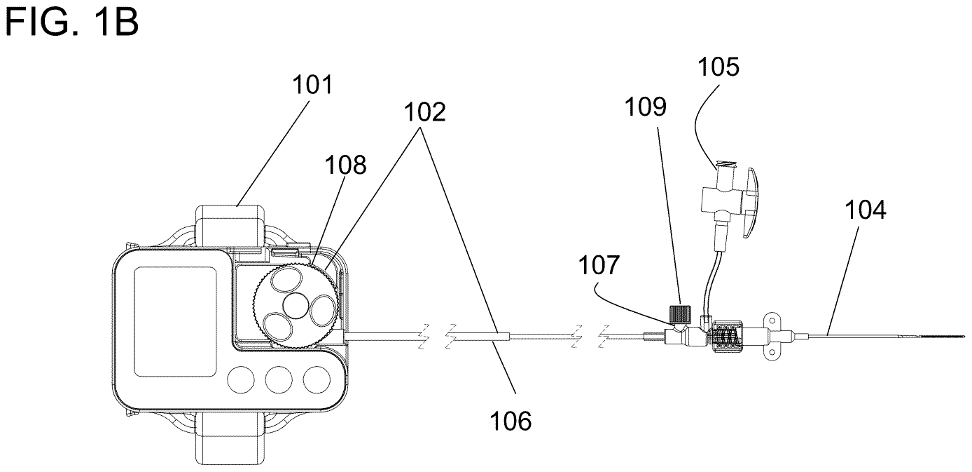

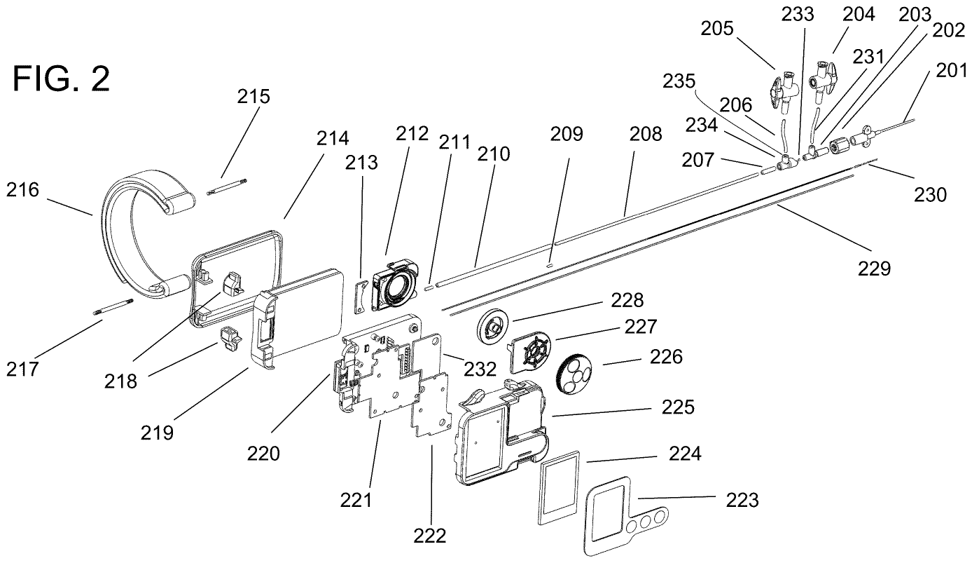

Referring to FIG. 1A, the intravascular monitoring system may include a measurement unit 101, a sensor line unit 102, a valve assembly (e.g., a plurality of valves such as stopcocks 103) with flow paths, and an access catheter 104, which may be inserted into a body (e.g., blood vessel). Instead of a plurality of valves, in some variations, the valve assembly may include a valve (e.g., a stopcock valve 105) and a port 107, as shown in FIGS. 1B and 1C. The port 107 may include a cap 109 releasably coupled to the port 107 that may be removed when, e.g., calibration of the one or more sensors is to be performed. FIG. 1B shows the port 107 covered by the cap 109, and FIG. 1C shows the cap 109 removed from the port 107. An elongate member (not shown) comprising one or more sensors (e.g., embedded therein) may be incorporated (e.g., slidably disposed therein) into the sensor line (line tubing) of the sensor line unit 102. As mentioned above, instead of the elongate member comprising the one or more sensors, in some variations, the one or more sensors may be coupled to the elongate member. The elongate member may be extended (advanced) from the sensor line unit 102 through the access catheter 104 and into the vessel to directly measure a physiological parameter (e.g., pressure) within the vessel. As shown in FIG. 3, the sensor line unit including a cartridge 302 may be disconnected from the measurement unit 301. The cartridge 302 may comprise a coupling member (e.g., a flexible tab 304) that may allow for securement of the cartridge 302 to the measurement unit 301. Although shown at the top of the cartridge 302, it is understood that tab 304 may be positioned on other portions of the cartridge 302, e.g., the sides or the bottom of the cartridge 302.

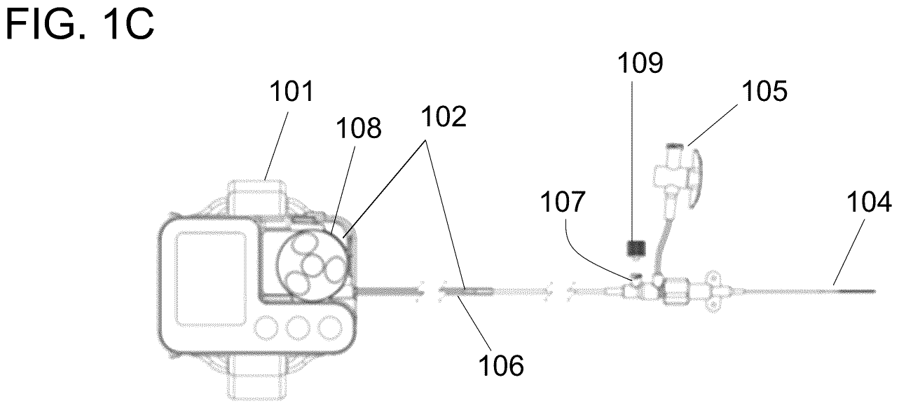

FIG. 2 shows an expanded view of the components that may be used to assemble the intravascular monitoring system. Referring to FIG. 2, an access catheter 201 may be configured for insertion into a blood vessel. The access catheter 201 may provide the elongate member with access into the blood vessel. Connectors 202, 203 may be configured to releasably couple the access catheter 201 to a portion of the valve assembly, e.g., stopcock valve 204. The valve assembly, which may include a plurality of connectors 202, 203, 234 and a seal 233, may provide a conduit for the elongate member 230 to pass through to the access catheter 201 while also providing a conduit to the line 231 that may lead to stopcock valve 204. A second piece of tubing 206 may lead to a second stopcock valve 205. The seal 233 may create a fluid tight seal between stopcock valves 204 and 205. Instead of being connected to second stopcock valve 205 via tubing 206, in some variations, a port 235 of the connector 234 may be covered by a cap (e.g., as shown in FIG. 1B).

When the cap is removed, the port may be used in calibrating the one or more sensors of the sensor line unit by exposing the sensors to the environment and/or to couple the port to a source of fluid and/or medication. Tubing sections 208 and 210 may form the main body of a sensor line (e.g., sensor line 106 of the sensor line unit 102 in FIGS. 1A and 1B) disposed between the cartridge body (e.g., cartridge body 108 in FIGS. 1A and 1B) of the sensor line unit 102 and the valve assembly. A strain relief member 207 may prevent kinking of tubing section 208 and may facilitate attachment to the connector 234. A protective sheath 229 may be placed over the elongate member 230 to contain and protect the electrical wires from the sensor running along the elongate member 230. A visual marker 209 may be attached to a proximal end of the elongate member 230 and distal end of transparent tubing 210 so that the position of the elongate member 230 may be tracked visually as the elongate member 230 is advanced or retracted during use. The connector 211 may aid in attachment of the catheter shaft to the cartridge body 212 of an actuation mechanism (e.g., for advancing and/or retracting the elongate member 230), and may also act as a stop for the visual marker 209. The connector 211 may also function as a strain relief for tubing 210.

Still referring to FIG. 2, a circuit board 213 may be incorporated into the cartridge body 212. The circuit board 213 may include connections and memory with cartridge/sensor information so that electrical and communication connections may be established when the cartridge body 213 is inserted into the remainder of the measurement unit. The elongate member 230 may be wound around a cartridge wheel 228, which may sit within the cartridge body 212. The wheel 228 may spin by rotating a grip wheel 226. When the grip wheel 226 is rotated, it may retract or advance the wire. A cartridge seal (not shown) may sit between the cartridge wheel 228 and the cartridge cover 227 creating a seal while allowing rotation. The housing of the measurement unit may be composed of a cover 220 and a housing body 225. One or more circuit boards 221, 222, and 232 may be mounted inside the housing. A removable (e.g., rechargeable) battery pack (not shown) may be contained in a battery housing including covers 214, 219. The battery housing may slide into a cartridge housing cover 220 and lock in place through interaction of buttons 218 with notches in the cartridge housing cover 220. A securing strap 216 may be connected to the measurement unit by pins 215, 217. A display screen 224 may be attached to a front panel 225 of the measurement unit for user input as well as display of data output from the measurement unit. A button membrane 223 may have individual buttons for user control of the measurement device and/or system.

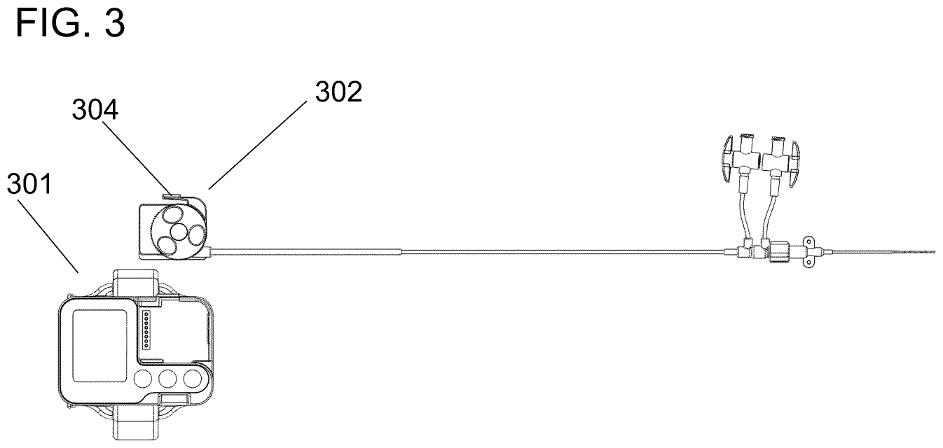

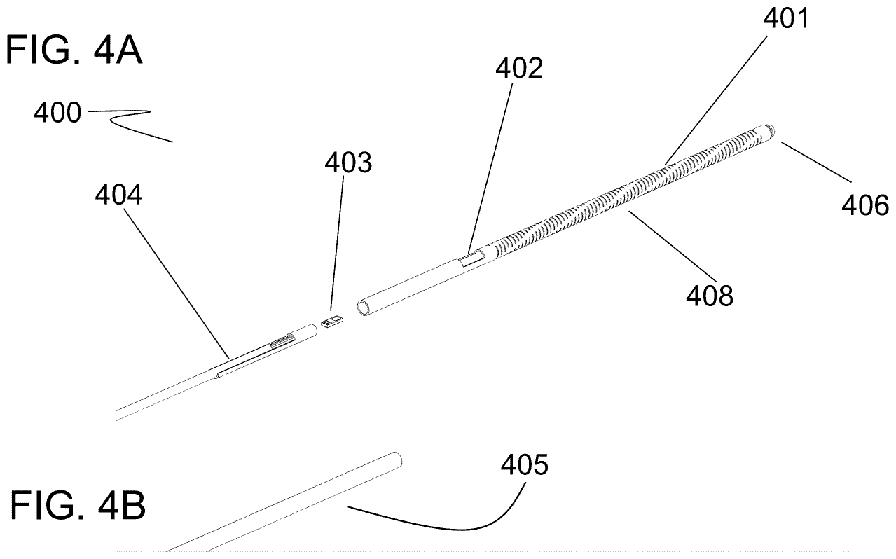

An exemplary elongate member including a sensor for measuring at least one physiological parameter is shown in FIG. 4A. Referring to FIG. 4A, an elongate member 400 may include a tube 401 including a lumen and an atraumatic tip 406. Laser markings 408 may be provided on all or a portion, e.g., a distal portion of the tube 401, to impart flexibility to the tube 401. A sensor window 402 may also be included in the tube 401 to expose a surface of the sensor 403 to the environment outside the elongate member 400. The sensor 403 may be mounted to a sensor holder 404, which may be inserted into the tube 401 to an extent that aligns the sensor 403 with the sensor window 402. An elongate member sheath 405, as shown in FIG. 4B, may be utilized to provide a layer of protection over the elongate member 400 and strengthen the joint between the sensor holder 404 and the tube 401.

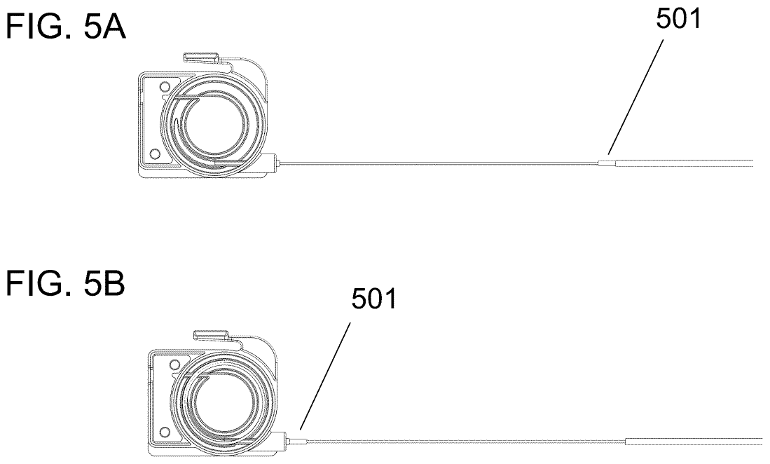

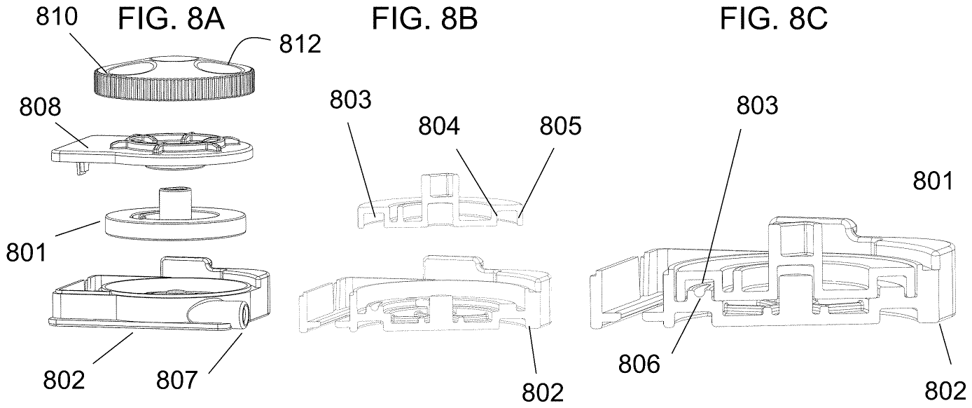

Further details of the cartridge body used to advance and retract an elongate member having one or more sensors thereon or therein are provided in FIGS. 8A to 8C. In FIG. 8A, the cartridge body includes a cartridge wheel 801 disposed between a cartridge housing 802 and a cartridge cover 808. A grip wheel 810 having one or more openings 812 for grasping by a user may be attached to the cover 808 to facilitate rotation of the wheel 801. In FIG. 8B, a cross-section of the cartridge wheel 801 and housing 802 separated from one another is provided to illustrate the space 803 between the cartridge wheel 801 and housing 802. The space 803 may be useful since it may allow for the elongate member (see FIG. 5) to be wound around the inside edge 804 of the cartridge wheel 801 during retraction of the elongate member and expanded to the outside edge 805 of the cartridge wheel 801 during advancement of the elongate member. Rotation against the inner edge 804 and outside edge 805 of the wheel may prevent the elongate member from binding (e.g., twisting, snagging on portions of the elongate member looped around the wheel, having uneven tension) during winding (retraction) and unwinding (advancement). In the variation shown in FIGS. 8A-8C, the elongate member may enter the wheel 801 from a face other than 804 or 805. For example, the elongate member may enter the wheel 801 via a bottom face 806, which may allow the elongate member to pass from the center of the space 803, below the wheel 801 and through a housing port 807.

The systems described herein may be configured to measure one or more physiological parameters, such as, but not limited to, blood pressure, mean arterial blood pressure, a pH level, a lactate level, a carbon dioxide level, and an oxygen level. In one variation, the at least one physiological parameter is blood pressure. The one or more physiological parameters may be measured while the one or more sensors is positioned within a body (e.g., within a blood vessel, e.g., an artery or a vein, or other body cavity).

### Auxiliary Sensor (Wearable Sensor)

In some variations, the systems for measuring physiological parameters of a patient may include at least a first sensor configured to measure at least one physiological parameter (e.g., blood pressure from within a blood vessel), and an auxiliary (e.g., wearable) sensor configured to calibrate the first sensor. In variations in which the first sensor is indwelling, the first sensor may be coupled to an elongate member of a sensor line unit, which may itself be releasably coupled to a measurement unit. In these variations, the first sensor may be configured to measure a physiological parameter, e.g., blood pressure, from inside a body (e.g., blood vessel such as an artery or a vein). In other variations, the first sensor may be a sensor configured to measure the physiological parameter from outside the patient's body, such as, for example, a blood pressure sensor of a conventional arterial line. In yet other variations, the system may include a plurality of first sensors, one or more of which is indwelling and one or more of which is external to a patient's body. In some variations, the first sensor may be a solid-state sensor. The wearable sensor may include an adhesive layer to aid in securing the wearable sensor to a portion of the patient's body (e.g., on the chest, at the level of the patient's heart). Any adhesive suitable for use on a patient's skin may be included in the adhesive layer. The wearable sensor may include a barometer. A single wearable sensor or multiple (e.g., two, three, or more) wearable sensors may be included in the systems.

The wearable sensor may reside within a compartment in the measurement unit until it is needed. After use, the wearable sensor may be placed back into the compartment, or if it is disposable, the wearable sensor may be discarded and another clean wearable sensor may be placed in the compartment. The compartment may have an electrical connection to “pair” the wearable sensor to one or more controllers (e.g., one or more processors) of the measurement unit and/or other devices. The “pairing” process may allow for the measurement unit and wearable sensor to share encryption keys and other credentials required for wireless communications.

### Kits

One or more sensor line units may be included in a kit, where the sensor line units may include an elongate member having one or more sensors along a length thereof configured to measure at least one physiological parameter from within a blood vessel. The sensor line units may also be configured to advance the elongate member to position the one or more sensors within the blood vessel, and retract the elongate member to position the one or more sensors outside of the blood vessel. The kits may further include any number of measurement units, valve assemblies, access catheters, connectors (with and without ports), caps for the ports, and auxiliary sensors (e.g., wearable sensors).

### Methods

Methods of measuring physiological parameters of a patient using the indwelling sensor systems are also described herein. In general, the one or more sensors may be placed in the body (e.g., inside of a blood vessel) using an elongate member. The one or more sensors may be mounted to, or embedded within, the elongate member, which may generally have an atraumatic tip at its distal end. A proximal end of the elongate member may be connected to a measurement unit that contains electronics to convert the data from the one or more sensors to a physiological parameter value, e.g., a pressure value. The measurement unit may be capable of displaying the physiological parameter values directly on a screen and/or it may send the data to a bedside monitor either wirelessly or via a wired connection. The measurement unit may have an interface so that the sensor may be controlled to zero and/or the baseline reading may be changed. The measurement unit may also include batteries, as previously described, so that the unit may work via battery power. In other variations, batteries may be omitted and the measurement unit may be powered via a wall outlet.

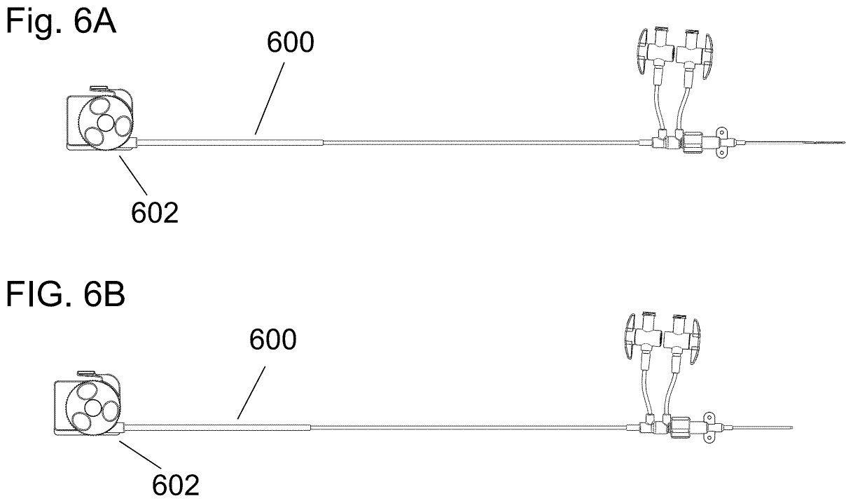

The sensor line units described herein may be configured to advance the elongate member to position the one or more sensors within the body (e.g., within a blood vessel), and retract the elongate member to position the one or more sensors outside of the body. As shown in FIGS. 5A and 5B, a visual marker may aid in tracking this advancement and retraction. For example, FIG. 5A illustrates the position of the visual marker 501 when the elongate member is advanced, and FIG. 5B shows the position of the visual marker 501 when the elongate member is retracted. In some variations, the visual marker 501 may also be structured as a physical stop that limits advancement and retraction of the elongate member. As shown in FIGS. 6A and 6B, a portion of an outer catheter/sheath may include a translucent or clear covering 600 extending from the sensor line unit 602 so that the visual marker 501 (see FIGS. 5A and 5B) is visible as it moves back and forth.

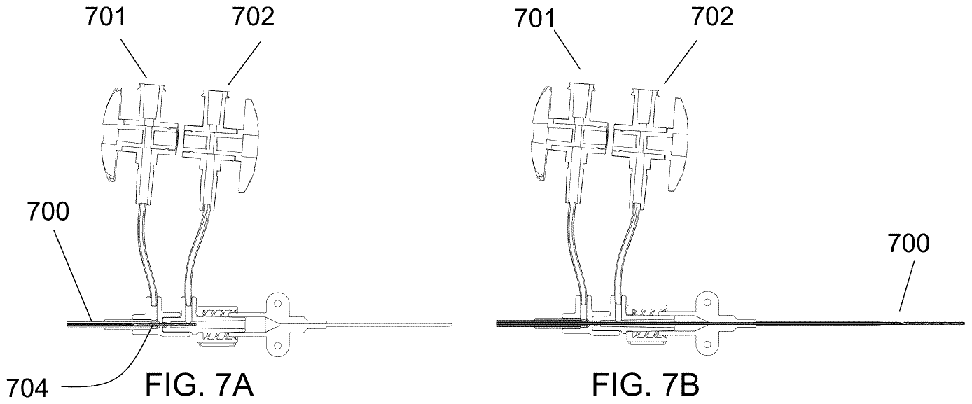

The systems described herein may include a valve assembly having at least a first valve and a second valve, or a first valve and a port, as previously mentioned. When the one or more sensors are placed in a blood vessel, a seal disposed between the first valve and the second valve or port may allow blood to be drawn from the first valve, e.g., when a distal tip of the elongate member is positioned (e.g., by retraction) between the first valve and the second valve or port. The second valve or port may be configured to calibrate the one or more sensors when the one or more sensors is positioned at the second valve or an uncapped port. For example, referring to FIG. 7A, when the elongate member 700 is retracted, the stopcock valve 701 may be opened so that the pressure in the environment outside is in direct communication with the sensor 704 within the system. This may allow the sensor 704 to be “zeroed” to atmosphere pressure if a zero point is required for the sensor 704 to be used. In this retracted configuration, the stopcock valve 702 may also be in direct communication with a fluid path to the system and blood may be withdrawn through the fluid path to stopcock valve 702 so that blood may be collected for analysis external to the system, for example, simultaneously with the sensor 704 being exposed to atmospheric pressure. In the retracted configuration, normal saline or other medications may also be provided through the stopcock valve 702 and to the patient. In this manner, the system is configured to allow for simultaneous calibration (or recalibration) of the sensor 704 and blood sampling, system flushing with a fluid, e.g., normal saline, and/or medication delivery. Additionally or alternatively, the access catheter could be flushed with a heparin solution to prevent blood from clotting within the catheter. When the elongate member 700 is advanced into a blood vessel, as shown in FIG. 7B, the fluid pathways from stopcock valves 701 and 702 are no longer open to the system, and thus, blood is not allowed to backup into the system.

In some variations, the elongate member including one or more sensors may be placed within an artery in the patient's wrist or in their leg, both of which are at a lower elevation than the patient's heart. For example, when placed within vasculature of the patient's wrist, the wrist may be resting on the operating table or bed. Given that the patient's heart may be several inches higher than their wrist, the blood pressure value reported by the one or more sensors may not accurately reflect the actual pressure in the aorta or other parts of the body.

Accordingly, in order to obtain a more accurate measurement of the central blood pressure, the system may include an auxiliary sensor (e.g., a second sensor) such as a wearable sensor (e.g., a body-worn sensor such as an adhesive patch). Both the wearable sensor and the measurement unit may include a barometric pressure sensor. When the system is started, the patch may be placed next to or in contact with the measurement unit, and the barometric pressure values may be read from the barometer in the measurement unit and the barometer in the wearable sensor. Given that at this point the wearable sensor and measurement unit are both at the same elevation, any differences in the values read from the barometers may be later factored out.

The wearable sensor may have either a wired or wireless connection (or both). The wireless connection may use a standard scheme such as Bluetooth, or other schemes such as UWB (Ultra-WideBand) technology, etc. There may be several modes of use for the wearable sensor. In the first mode, after the system has been started and the zeroing completed, the wearable sensor may be placed on the patient's chest or clothing at a point that is approximately at the level of the patient's heart. The barometric pressure values may again be read from the two barometers, and the change in the values (from the initial measurements) may be used to calculate the height difference between the one or more sensors within the vasculature and the wearable sensor. The height difference may then be used to calculate the effective blood pressure equivalent, and that amount may in turn be used to adjust the values reported to the user. For example, if the wearable sensor is found to be about 7 cm above the one or more first sensors (e.g., indwelling sensors), the reported blood pressure may be adjusted based on that difference.

In a second mode, the wearable sensor may instead be placed on top of the chest. This variation may be used in situations where it may not be feasible to place the wearable sensor at the exact level of the heart. In this second mode, the heart level may be estimated to be at some point between the elevation of the one or more indwelling sensors and the wearable sensor. For example, if the elevation of the wearable sensor is about 12 cm higher than the one or more first sensors (e.g., indwelling sensors), the heart may be estimated to be about 6 cm (12 cm/2 ) above the heart.

In a third mode, calibration may be performed with the one or more first sensors and wearable sensor at the level of the patient's heart. In this case, a medical professional (e.g., nurse, physician, or surgeon) may have to lift the patient's wrist to the level of the heart. As in the other modes, the wearable sensor may be placed at the level of the heart or on top of the chest.

In all of these modes, the barometric values of the wearable sensor and measurement unit may be read periodically, such as, for example, at a rate of once per second. This allows for accurate reporting of the patient's blood pressure if the one or more of the first sensors (e.g., indwelling sensors) is moved, such as when a surgeon needs to move the patient's arm with an indwelling sensor during surgery, if the patient is rolled onto their side, or if the head of the patient's bed is raised. For example, if the measurement unit is attached to the patient's right wrist and the patient is rolled onto their left side, the one or more indwelling sensors may be positioned at a point higher than then the wearable sensor. This condition may be detected because the barometric pressure of the measurement unit will be less than the pressure indicated by the wearable sensor, and the calculations for the pressure at the heart will be adjusted accordingly. If the wearable sensor was attached to the patient's chest (e.g., as in the second mode described above), then the heart level may be the same level as the wearable sensor (not ½ the difference between the indwelling sensors and the wearable sensor). In another variation where the wearable sensor is placed on the left side of the patient's chest (e.g., as might be done for the first mode described above), and the patient is rolled onto their left side, the patient's wrist (and the measurement unit) may end up positioned near the patient's hip and the wearable sensor close to or touching the operating table or bed. In this instance, the wearable sensor may no longer be at the patient's heart level. In this case, the heart may be estimated to be approximately ½ the height difference between the wearable sensor and the wrist level measurement unit.

In addition to conveying barometric pressure to the measurement unit, the wearable sensor may also convey other data. For example, other data that may be transmitted may relate to the operating state of the measurement unit, such as the battery charge level. In some variations, the wearable sensor may also include other physiological sensors, such as accelerometers (for measurement movement of the patient's chest and calculating respiration rate), ECG sensors, temperature sensors, an ohmmeter (for measuring skin impedance), and microphones (for a stethoscope function).

Several wearable sensors may be used concurrently, e.g., one placed on the center of the chest, one on the side of the chest, and one on the back of the chest. In some variations, the wearable sensor may also include an actuator, such as a small motor or audio speaker. These actuators may create vibrational patterns in the body that may be measured elsewhere, and may assist other sensors in determining their location. Another type of actuator may cause a small high-frequency alternating voltage that may be measured by the sensor in the measurement unit or by other sensors. This may be used not only to provide positive proof of connectivity, but may also be used to detect build-up of fluids in the chest.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated.

1. A system for measuring one or more physiological parameters of a patient comprising:
a measurement unit comprising a housing; and
a sensor line unit releasably coupled to the housing, the sensor line unit comprising an elongate member having one or more sensors along a length thereof configured to measure the one or more physiological parameters from within a blood vessel,
wherein the sensor line unit is configured to advance the elongate member to position the one or more sensors within the blood vessel, and retract the elongate member to position the one or more sensors outside of the blood vessel.

2. The system of claim 1, further comprising a valve assembly coupled to a sheath disposed over the elongate member.

3. The system of claim 2, wherein the valve assembly comprises a connector and one or more of a first valve or a second valve.

4. The system of claim 1, further comprising one or more connectors, the one or more connectors comprising one or ports configured to calibrate the one or more sensors.

5. The system of claim 3, wherein the valve assembly comprises a seal between the first valve and a port.

6. The system of claim 5, wherein the port is configured for coupling to a reservoir containing a fluid.

7. The system of claim 6, wherein the fluid comprises a medication, normal saline, or a heparin solution.

8.-10. (canceled)

11. The system of claim 3, wherein the first valve is configured to withdraw a blood sample when a distal tip of the elongate member is positioned between the first and second valves.

12. The system of claim 2, further comprising an access catheter coupled to the valve assembly.

13. (canceled)

14. The system of claim 2, wherein a proximal portion of the sheath comprises a translucent material.

15. The system of claim 1, wherein the measurement unit is reusable.

16. (canceled)

17. The system of claim 1, wherein the measurement unit comprises a display configured to visually present data relating to the measured the one or more physiological parameters, instructions for setup of the system, an error in function of the measurement unit, sensor line unit, one or more sensors, or a combination thereof.

18. The system of claim 1, wherein the measurement unit comprises a strap configured to releasably attach the measurement unit to a limb of the patient.

19. (canceled)

20. The system of claim 1, wherein the measurement unit comprises a controller configured to wirelessly communicate the measured one or more physiological parameters with one or more devices.

21. The system of claim 1, wherein the one or more physiological parameters is selected from: blood pressure, mean arterial blood pressure, a pH level, a lactate level, a carbon dioxide level, and an oxygen level.

22. (canceled)

23. The system of claim 1, wherein the one or more sensors comprises a solid-state sensor.

24. The system of claim 1, wherein the sensor line unit is disposable.

25. The system of claim 1, wherein the sensor line unit comprises a cartridge configured to couple with the housing of the measurement unit, the cartridge comprising an actuator configured to advance and retract the elongate member.

26. The system of claim 25, wherein actuator comprises a wheel, an actuator housing, and space therebetween configured to prevent binding of the elongate member during one or more of retraction or advancement of the elongate member.

27.-29. (canceled)

30. The system of claim 1, wherein the elongate member comprises a window configured to expose the sensor to blood within the blood vessel.

31. The system of claim 1, wherein the blood vessel is an artery or a vein.

32. The system of claim 1, further comprising a body-worn sensor.

33. (canceled)

34. A method of measuring one or more physiological parameters of a patient comprising:
positioning an elongate member of a sensor line unit comprising one or more sensors into a blood vessel;
coupling the sensor line unit to a measurement unit; and
measuring one or more physiological parameters of the patient,
wherein the elongate member is advanced to position the one or more sensors within the blood vessel and retracted to position the one or more sensors outside of the blood vessel.

35.-55. (canceled)

56. A kit comprising a plurality of sensor line units of claim 1.

57.-60. (canceled)

61. A system for calibrating a blood pressure sensor comprising:
a first sensor configured to measure a blood pressure of a patient;
a wearable sensor comprising a barometer; and
a processor configured to calibrate the measured blood pressure from the first sensor based on data from the wearable sensor.

62.-63. (canceled)
